(19) United States
(12) Patent Application Publication
(10) Pub. No.: US 2026/0245693 A1
(43) Pub. Date: Aug. 20, 2026

(54) SYSTEMS AND METHODS FOR ACCURATELY DETERMINING INDICATIONS ASSOCIATED WITH PRESCRIPTION CLAIM DATA

(71) Applicant: OPTUM, INC., Minnetonka, MN (US)

(72) Inventors: Jun Han, New Hyde Park, NY (US); Niall William Heavey, Dublin (IE); Suhit Dey, Dublin (IE); Robert Elliott Tillman, Long Island City, NY (US)

(21) Appl. No.: 19/233,862

(22) Filed: Jun. 10, 2025

Related U.S. Application Data
(60) Provisional application No. 63/759,954, filed on Feb. 18, 2025.

(51) Int. Cl. G16H 20/10; G06N 20/20; G16H 10/60

(52) U.S. Cl. CPC G16H 20/10; G06N 20/20; G16H 10/60

(57) ABSTRACT

The disclosure is directed to determining one or more indications for a prescribed medication that should be present in claim data for an individual, e.g., when an indication is missing from the claim data. To this end, the disclosed techniques generate a set of features that includes a first feature vector reflective of frequencies with which a provider has previously prescribed the medication, and a second feature vector reflective of frequencies with which the medication has been prescribed to the individual, with the feature vectors being in vector spaces having dimensions that correspond to different candidate indications for the medication. The techniques may also include generating an additional feature based on structured metadata related to the claim, and/or the techniques may use an ensemble machine-learned model to select one of the candidate indication codes (e.g., to confirm the claim indications or provide a missing indication).

### CROSS-REFERENCE TO RELATED APPLICATIONS

This application claims the benefit of U.S. Provisional Patent App. No. 63/759,954, filed on Feb. 18, 2025, the entire disclosure of which is hereby incorporated herein by reference.

### FIELD

Generally, the present disclosure relates to processing medical claim data. More specifically, the techniques of this disclosure relate to the use of machine-learned model(s) to infer claim information that correctly specifies the indication(s) for a particular medication.

### BACKGROUND

Typically, medical providers generate and submit medical claims for processing by a medical insurer. The claims, however, are often incomplete, causing delays or mistakes in processing. In the case of prescription for medicines, a missing indication can not only lead to delays in claim processing but also affect pricing. Furthermore, manufacturers of medicines rely on accurate indication data for a variety of decisions pertaining to marketing, production, and distribution of their products. The problem is exacerbated by the fact that a given medicine may be indicated for a variety of diagnoses. Accurately assessing how often a medicine is prescribed for each diagnosis, as well as accurately processing medical claims for the prescriptions, remains challenging.

### BRIEF DESCRIPTION OF THE DRAWINGS

The Figures described below depict preferred embodiments for purposes of illustration only. One skilled in the art will readily recognize from the following discussion that alternative embodiments of the systems and methods illustrated herein may be employed without departing from the principles of the disclosure described herein.

FIG. 1 depicts an example computing environment in which various embodiments of the present disclosure may be implemented.

FIG. 2 depicts an example data flow for determining or selecting an indication code that should be associated with a claim in accordance with various embodiments described herein.

FIG. 3 depicts an example feature generator, in accordance with various embodiments described herein.

FIG. 4 depicts an example ensemble machine-learned model, in accordance with various embodiments described herein.

FIGS. 5A and 5B depict a flow diagram representing an example computer-implemented method, in accordance with various embodiments described herein.

### DETAILED DESCRIPTION

Broadly speaking, the techniques of the present disclosure relate to processing medical claim data and determining accurate indications for claim data. In the case of processing medical claim data for medications prescribed and/or prescriptions filled by providers, an indication for a given prescription (i.e., the reason for prescription and/or associated diagnosis) is too often missing. When a claim is filled out correctly, the indication can typically be found based on the billed diagnosis code on a claim. However, the indication or diagnosis codes may be missing because claims are not always fully filled out. For example, a pharmacist may not fill out the indication or diagnosis code portion of the claim, leaving it blank. The number of impacted claims typically varies depending on the class of the medication. The total percentage of missing diagnosis codes for immunomodulators, for example, is approximately 30% and rises to over 65% for antidiabetics. Occasionally, the claim may be miscoded, and a claim processor (e.g., a responsible person or a claim processing system) may deem that a correction is warranted.

In most cases, the claim processor cannot accurately infer the indication solely from the prescription because a given medication can be prescribed for a variety of indications, and only a subset of those indications may be appropriate in any given instance. For example, Humira® is approved in the United States by the Food and Drug Administration for nine indications including rheumatoid arthritis, Crohn's disease, etc. A dosage and duration of a prescription may carry some small amount of information regarding the indication, but without additional data, filling in a missing indication is tantamount to a guess.

In some scenarios, a claim processing system may obtain additional information to aid in claim processing. For example, an individual receiving a prescription (e.g., a patient) may have a medical history that the claim processing system can obtain. In the simplest case, a record may exist of a provider having prescribed or having filled a prescription of a medication, and the system can access the associated indication. A simple business rule may include generating a missing indication by assigning a previous indication in lieu of a missing one. Practice shows, however, that this rule leads to a substantial number of errors. Other similar rule-based approaches have not yielded satisfactory results.

The techniques of this disclosure can engineer concise (low data volume) and informative (highly inferential or predictive) features that enable a machine-learned model to efficiently generate an accurate indication for a prescribed medication, e.g., when the indication is missing from claim data. For each prescribed medicine, there is a set of possible indications. The techniques of this disclosure combine claim data with other data to infer which of the set of possible indications is/are correct. The techniques use one or more machine-learned models, the input for which includes at least two vectors that are generated based on frequencies of prior indications. In particular, a first vector is generated using the indication history of the medicine for the specific medical provider prescribing the medication, and a second vector is generated using the indication history of the medicine for the specific individual for whom the medicine is prescribed. Additionally, the techniques may generate one or more additional features, such as a third feature based on claim metadata relating to the individual receiving the prescription (e.g., age, gender, etc.) and/or relating to details of the prescription that describe the course of treatment (e.g., dosage, duration, number of refills, etc.). These techniques can dramatically improve the accuracy of generating missing indications, and have achieved, in testing, percentage success rates in the upper nineties. Furthermore, by efficiently encoding frequencies with which a medication has been prescribed by a medical service provider and frequencies with which the medication has been prescribed to the individual associated with the claim, the engineered features of this disclosure enable efficient use of computing resources. In particular, the techniques of this disclosure distill within the feature vectors (and possibly other features) information that is highly relevant to accurately determining the correct indication code(s) and, consequently, avoid the need for machine-learned model(s) to process larger data objects. Moreover, by generating concise feature vectors instead of including a larger volume of less relevant/predictive data, the disclosed techniques can reduce the likelihood that the machine-learned model(s) will generate inaccurate outputs (e.g., due to spurious data that “confuses” the model(s)). In some embodiments, the disclosed techniques further include an ensemble machine-learned model architecture specifically configured to infer or predict the correct indication, e.g., when an indication is missing from claim data.

Of course, it should be appreciated that the advantages and technical improvements described above and elsewhere herein are not the only advantages and/or technical improvements that may be realized using the techniques described herein. Other advantages and/or technical improvements to the functioning of a computer itself or other technologies or technical fields may be apparent to one of ordinary skill in the art. Moreover, while described herein primarily in the health care claims context, the techniques described herein may be readily applied in any suitable field for any suitable purpose.

### Example Computing Environment Including a Computing System

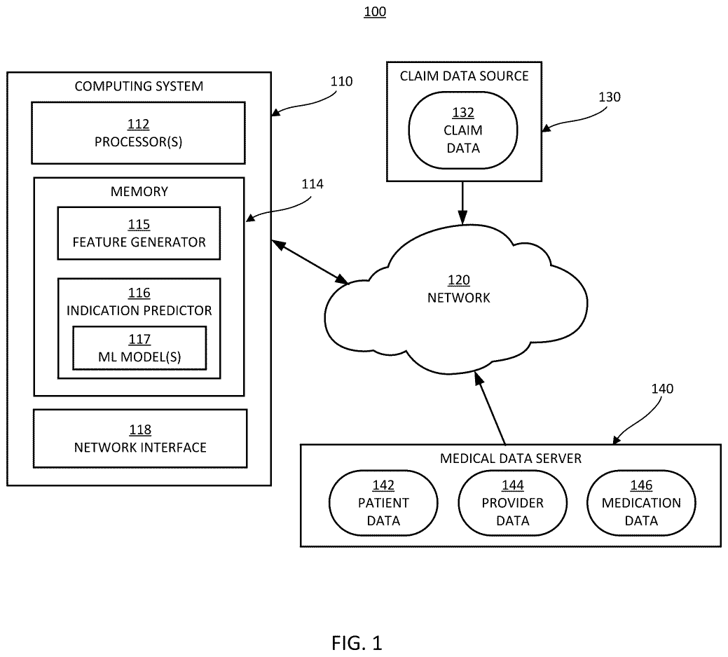

FIG. 1 depicts an example computing environment 100 in which various techniques of the present disclosure may be implemented. The computing environment 100 includes a computing system 110, which may perform at least some of the techniques of this disclosure. The computing system 110 includes a processor(s) 112 communicatively coupled to memory 114, which may store feature generator 115 and indication predictor 116 algorithms, and communicatively coupled to a network interface 118. The indication predictor 116 may include one or more machine-learned models 117. The computing system 110 is communicatively coupled, by way of the network interface 118, to a network 120.

The processor(s) 112 may include any suitable number of processors and/or processor types. In some examples, the processor(s) 112 include one or more central processing units (CPUs), one or more graphics processing units (GPUs), one or more tensor processing units (TPUs), one or more field-programmable gate arrays (FPGAs), one or more application-specific integrated circuits (ASICs), and/or the like. Generally, the processor(s) 112 comprise hardware configured to execute processor-executable code/instructions stored in the memory 114.

The memory 114 may include any suitable memory type(s), including one or more volatile memories (e.g., dynamic and/or static random-access memory (RAM)) and/or non-volatile memories (e.g., read-only memory (ROM), erasable programmable ROM (EPROM), electrically EROM (EEROM), NAND flash, and/or solid state drive(s) (SSD(s))), all or any of which are examples of non-transitory, computer-readable media. In some examples, the memory 114 stores one or more of: an operating system; one or more software components (e.g., firmware, application(s), binary, source code, executable instructions, machine-learned model(s)); transient data and/or code loaded and/or operated on by one or more software component(s); and/or other suitable components/data).

The computing environment 100 also includes a claim data source 130. The claim data source 130 may be a server or another suitable computing device configured to generate and/or store claim data 132. For example, the claim data source 130 may be a computing device (e.g., a desktop computer, a notebook computer, a tablet, a smartphone, etc.), and a health care provider may use the claim data source 130 to generate the claim data 132 and send the claim data 132, by way of the network 120, to the computing system 110. In other embodiments, the claim data source 130 may be a server configured to store and/or aggregate the claim data 132 generated by a health care provider (e.g., by way of a computing device). The computing system 110 may request and/or procure the claim data 132 from the claim data source 130 by way of the network 120.

The example computing environment 100 additionally includes a medical data server 140 communicatively connected to the network 120. The medical data server 140 is configured to store patient data 142, provider data 144, and medication data 146 in one or more databases. By way of the network 120, data (e.g., patient data 142, provider data 144, medication data 146) stored on the medical data server 140 may be available for processing by the computing system 110. In some embodiments, a collection of medical data servers may store any portion of the patient data 142, the provider data 144, and the medication data 146. That is, the data used by the computing system 110 may be suitably distributed and/or replicated across any suitable collection of servers. Furthermore, the computing system 110 may be communicatively connected to one or more data storage devices storing at least portions of the patient data 142, the provider data 144 and the medication data 146.

It should be noted that in different embodiments components of the computing environment 100 differ from what is depicted in FIG. 1, and/or may be combined in different ways. For example, the computing system 110 may include the claim data source 130 and/or the medical data server 140. Additionally or alternatively, the medical data server 140 may include the claim data source 130. Furthermore, the computing system 110 may include distributed systems, devices, and/or components (e.g., connected by way of the network 120) configured to implement the techniques of this disclosure. For example, the computing system 110 may include components in a cloud environment configured to train and/or run machine-learned models of this disclosure. Still furthermore, the computing system 110 may include and/or be in communicative connection with one or more input devices (e.g., scanners, tablets, etc.) configured to digitize and/or change the digital format of claim data 132.

In operation, the computing system 110 may receive (e.g., via the network 120 and from the claim data source 130) the claim data 132 for a medication prescribed by a provider for an individual. The claim data 132 may be structured data generated by a provider of medical services (e.g., a doctor, a nurse-practitioner, a pharmacist, etc.) or an agent of the provider (e.g., an assistant, a clerk, etc.). The provider or the agent may generate the claim data 132 by way of a computing device (e.g., claim data source 130 or another computing device or system) and, for example, using a digital form with defined fields. The fields may include the name and age of a patient, medication name, quantity, and dosage, and an indication (e.g., a medical diagnosis and/or an associated code) for the prescription. In some embodiments, the claim data 132 may be generated by a provider and, subsequently, stored in a database (e.g., the database server acting as the claim data source 130). On the other hand, the claim data 132 need not be in a digital form or, when in a digital form, need not be structured. For example, the claim data 132 may be a printed form that was filled out by hand or a digital scan of such form. Extracting the data from the form may require that the computing system 110 classify and process the form (e.g., using optical character recognition (OCR) techniques) to extract relevant portions of the claim data 132.

An indication for a medication can be an important portion of the claim data 132. However, such indications can sometimes be missing. For the purpose of this disclosure, an indication is considered “missing” regardless of whether the indication is incorrectly identified in the claims data (e.g., the wrong code was used) or the indication is simply absent from the claims data (e.g., absent from a portion or portions of one or more forms, one or more digital fields, one or more portions of a structured string, etc., that is/are expected to contain the indication(s)) with no other incorrect indication being included in its stead. An indication, which may be a diagnosis identified by a medical service provider, may be identified with a respective code. For example, an alpha-numeric code associated with a medical condition or a disease may accompany a prescription and serve as the indication. In some examples, the medical condition or disease name may be explicitly stated in claim data (e.g., the claim data 132). In this disclosure, the terms “indication,” “indication code,” and “diagnosis” may be used interchangeably, as they refer to the same information associated with a prescription.

As discussed above, an indication (or, as discussed, a correct indication) may be missing from the claim data 132. The task of identifying the correct indication is complicated by the fact that a particular medication may have a variety of different indications, as discussed above. Generally, the feature generator 115 and the indication predictor 116, when executed by the processor(s) 112, determine (e.g., infer or predict) the correct indication associated with the claim data and generate a data object indicative of the correct indication code (or, in some embodiments and/or scenarios, the set of multiple indication codes). The feature generator 115, when executed by the processor(s) 112, efficiently generates features that, when used as inputs to one or more machine-learned models included in (e.g., machine-learned model(s) 117) or called by the indication predictor 116, result in an accurate determination of the indication that should be associated with the claim data 132. The indication predictor 116 may call on machine-learned models stored in the memory 114 (e.g., machine-learned model(s) 117) within or outside of the indication predictor 116. In some embodiments, the indication predictor 116 may be communicatively coupled by way of the network 120 to a remote server that stores and executes the one or more machine-learned models. One or more additional components (e.g., algorithms, applications, etc.) may also be stored in the memory 114, and may obtain data to support the operation of the feature generator 115. Furthermore, one or more additional components stored in the memory 114, when executed by the processor(s) 112, may process the output of the indication predictor 116. Feature generator 115, indication predictor 116, and other components that may be stored in the memory 114 are described below with reference to the configuration and operation of the computing system 110.

The computing system 110 may be configured to determine the medication associated with the claim data 132. To that end, the computing system 110 may determine the field or string associated with the medication within the claim data 132. In some embodiments, the computing system 110 may use OCR techniques to determine the medication in a digital scan or otherwise obtained digital image of a filled-out form associated with the claim data 132.

The computing system 110 may be configured to obtain a set of candidate indication codes associated with the determined medication (i.e., the medication associated with the claim data 132). For example, the computing system 110 may send, by way of the network 120, a query to the medical data server 140, requesting data associated with the determined medication from the medication data 146. That is, the requested query output may include the set of candidate indication codes associated with a medication key used in the query, where the medication key corresponds to the identified medication associated with the claim data 132. The medical data server 140 may be configured to ensure that the medication data 146 stored on the medical data server 140 reflects current indications or indication codes (e.g., approved by a regulatory agency, used in past prescriptions, and/or identified in medical literature), such that the set of candidate indication codes obtained by the computing system 110 reflects the most current set of candidate indications or indication codes for the obtained claim data.

The computing system 110 may be configured to generate (e.g., compute/create/modify/update/etc., by the indication predictor 116 or by processing the output of the indication predictor 116) a data object indicative of one indication code, of the set of candidate indication codes, that should be (e.g., is most likely) associated with the claim data. Furthermore, the computing system 110, based on the output of the indication predictor 116, may generate a confidence score associated with the indicated code of the set of candidate indication codes, and include an indication of the confidence score (e.g., a number between zero and one or any other suitable indication) in the data object. Additionally or alternatively, the computing system 110 may generate a distinct data object indicative of the confidence of the indicated/selected indication code. In some embodiments and/or scenarios, the computing system 110 may indicate/select (e.g., based on the output of the indication predictor 116) a subset of two or more of the set of the candidate indication codes and compute respective likelihoods or confidence scores indicative of likelihoods that respective indication codes are associated with the claim data. The selected subset may include the entirety of the set of candidate indication codes. The computing system 110 may select an indication code based on the highest computed respective likelihood or confidence score (e.g., computed by the indication predictor 116), for example. In some embodiments, however, an input from a claim processor and/or other prior data factors into the selection, resulting in selecting an indication code different from the indication code with the highest confidence score (e.g., as computed by one or more machine-learned models). That is, the computing system 110 may select the indication code based at least in part on the computed confidence score.

To determine or select one or more indications from the set of candidate indications, the indication predictor 116 may use one or more machine-learned models stored at computing system 110 (e.g., machine-learned model(s) 117) or remotely call one or more such machine-learned models. Moreover, the feature generator 115 may generate a plurality of features as model input and the indication predictor 116 may cause the model input to be applied to one or more machine-learned models. In some embodiments, one or more of the machine-learned models may run on the computing system 110 (e.g., on processor(s) 112 executing the instructions of the indication predictor 116). Additionally or alternatively, the computing system 110, executing the instructions of the indication predictor 116, may generate the model input and send (e.g., by way of the network 120) the model input to a remote computing system hosting/running/etc. at least some of the machine-learned models. The remote computing system may, in turn, send output of the remote model to the computing system 110, where the output may be processed by the indication predictor 116. The computing system 110 may then generate the data object indicative of the indication code of the set of candidate indication codes and/or one or more confidence scores, as discussed above.

The machine-learned models used by the computing system 110 may include an ensemble model which includes multiple machine-learned models, the outputs of which may be combined to generate the aggregate output. Generally, the component models of the ensemble model may include decision-tree-based models, such as a random forest model, a convolutional neural network (CNN), a support vector machine (SVM), or any other suitable machine-learned model may take as an input the feature vector and output an indication code selection and, possibly, a score indicative of confidence in the selection. The outputs of the component models may be combined (e.g., as a weighted sum) to select the indication code from the set of candidate indications.

Features created by the computing system 110 according to the techniques of this disclosure can be used to accurately and efficiently (e.g., in terms of computing resources) select an indication code from the set of candidate indication codes. In particular, the computing system 110, by executing the instructions of the feature generator 115, generates multiple features, including two feature vectors. The first feature vector is indicative of frequencies with which respective candidate indication codes have been used by the provider. The second feature vector is indicative of frequencies with which respective candidate indication codes have been used for the individual for whom the medication of the claim data is prescribed. To generate the feature vectors, the computing system 110 may obtain prescription history data of the provider (e.g., from provider data 144) and the prescription history of the individual (e.g., from patient data 142) for whom the medication is prescribed. From these records, the feature generator may generate two vectors each having a number of elements corresponding to the number of possible candidate indications. The number of possible indications may be, for example, 2, 3, 5, 7, 10, etc., depending on approved uses for the medication. A given provider, based on their specialization or practice preferences, may favor certain indications. Therefore, the provider history for prescribing the medication may add information from which a missing indication may be inferred. A given person for whom a medication is prescribed may have a persistent condition for which the medication is indicated. This condition may be reflected in a prescription history. Therefore, prescription history of the individual adds additional information from which a missing indication may be inferred. It should be noted, however, that an individual may receive the same prescription for two different conditions at two different times. To help avoid errors based on such occurrences, the computing system 110 generates and uses both feature vectors rather than just the individual-specific feature vector. An example for generating feature vectors is outlined below.

In one example, a medication X may have three indications: A, B and C. Claim data for an individual to whom the medication X is prescribed may have a missing indication. However, the history of previous claims for the individual may include information that enables computing system 110 to determine frequencies with which the medication X has been prescribed to the individual. For instance, the individual may have been prescribed the medication X three times for indication A, once for indication B, and never for indication C, and the provider who wrote the prescription may have previously prescribed the medication X with the following distribution: 15 times for indication A, 60 times for indication B, and 75 times for indication C. The computing system 110 may then generate, using the feature generator 115, the first and the second feature vectors using the prescription history above. The first and second feature vectors may be three-dimensional vectors, with each dimension corresponding to a possible indication. That is, for a medication that has nine possible indications, the first and the second feature vector may each have nine dimensions.

Returning to the three-dimensional vector example above, using the feature generator 115, the computing system 110 may assign magnitudes to each of some or all of the dimensions of the first feature vector based on the frequencies with which the medication X has been prescribed by the provider. In one example, the computing system 110 may assign magnitudes in proportion to the frequencies. Accordingly, the computing system 110 may compute the first feature vector to be (0.1, 0.4, 0.5). For the first dimension, the magnitude of 0.1 is equal to 15 divided by 150, reflective of the fact that 15 of 150 previous prescriptions by the provider were for indication A. For the second dimension, the magnitude of 0.4 is equal to 60 divided by 150, reflective of the fact that 60 of 150 previous prescriptions by the provider were for indication B. For the third dimension, the magnitude of 0.5 is equal to 75 divided by 150, reflective of the fact that 75 of 150 previous prescriptions for the individual were for indication C. Similarly, the computing system 110 may assign magnitudes to each of some or all of the dimensions of the second feature vector based on the frequencies with which the medication X has been prescribed to the individual, e.g., in proportion to the respective frequencies. Accordingly, the computing system 110 may compute the second feature vector to be (0.75, 0.25, 0). For the first dimension, the magnitude of 0.75 is equal to 3 divided by 4, reflective of the fact that three of four previous prescriptions to the individual were for indication A. For the second dimension, the magnitude of 0.25 is equal to 1 divided by 4, reflective of the fact that one of four previous prescriptions for the individual were for indication B. For the third dimension, the magnitude of 0 is reflective of the fact that none of the previous prescriptions for the individual were for indication C. In the present example, the computing system 110 assigned dimension values to the feature vectors in a manner that normalizes the sum of all dimension magnitudes to unity. Generally, a feature vector need not be normalized in this manner.

In the above example, the first feature vector is more suggestive of indication B, while the second feature vector is more suggestive of indication A. Generally, the computing system 110 uses both feature vectors to account for both the provider and the individual prescription history and make a statistically more accurate determination than if either of the feature vectors were to be used by itself. Using the indication predictor 116, the computing system 110 may send each of the feature vectors to a separate machine-learned model and then combine the outputs of the models. In other embodiments, a single machine-learned model may take both feature vectors as inputs to predict the indication.

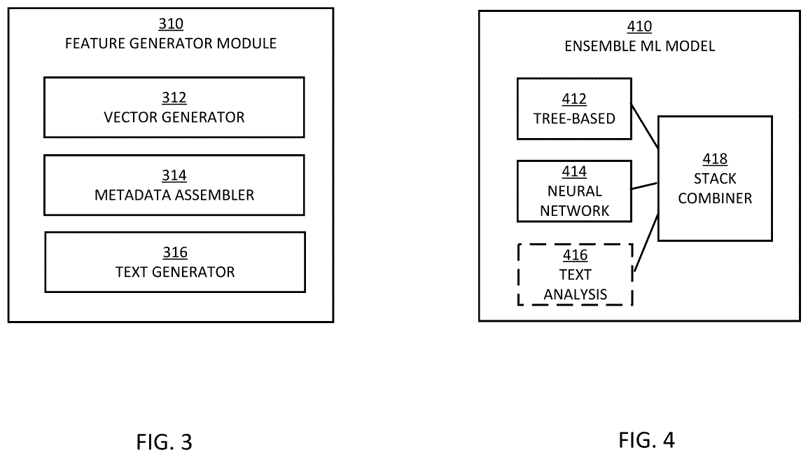

It should be noted that feature vector component magnitudes (i.e., magnitudes of vector components along each dimension of a vector space) need not be strictly in proportion to respective frequencies. In some embodiments, the computing system 110 uses a recency factor to assign magnitudes to the feature vectors. In the example above, the one prescription with the indication B for the individual may be recent (e.g., previous month, three months, a year, etc.), while the three prescriptions with the indication A may be older (e.g., two, five, ten years ago, etc.). In computing feature vector component magnitudes, the computing system 110 may weigh more recent prescriptions more highly. The provider, too, may dynamically adjust, over time, frequencies of prescribing a given medication for different indications. As with prescriptions to the individual, the computing system 110 may weigh more recent prescriptions more highly, as discussed below with reference to FIG. 3.

In some embodiments, in addition to the two feature vectors described above, the computing system 110 generates a third feature that is also to be used as input to a machine-learned model, at least in part by extracting structured metadata from the claim data 132 and/or another suitable source. The structured metadata used in the third feature may include, for example, one or more characteristics of the individual for whom the medication is prescribed. The one or more characteristics may include age, gender, height, weight, test results, known medical conditions, and/or other suitable characteristics. Additionally or alternatively, the structured metadata used in the third feature may include one or more characteristics of the medication that is/are indicative, for example, of quantity (e.g., number of tablets or duration of course) and/or dosage. Additionally or alternatively, the structured metadata used in the third feature may include characteristics of the provider including, for example, specialization, affiliations, and/or years in practice.

Before the features described above are generated, the computing system 110 may obtain suitable data from the medical data server 140, the claim data source 130, and/or other suitable data repositories. The computing system 110 may receive the claim data 132 for the medication prescribed by the provider for the individual from the claim data source 130. The computing system 110 may use the medication identified in the claim data 132 to obtain the set of candidate indication codes from the medication data 146 stored at the medical data server 140. The computing system 110 may obtain data indicative of frequencies with which respective candidate indication codes have been used by the provider from the provider data 144 stored at the medical data server 140. The computing system 110 may obtain data indicative of frequencies with which respective candidate indication codes have been used for the individual from the patient data 142 stored at the medical data server 140.

Using the data obtained from the claim data source 130 and from the medical data server 140, the computing system 110 may generate the machine-learned model input features described above. The computing system 110 may generate, using one or more machine-learned models, a data object indicative of one of the indication code that is in the set of candidate indication codes. That is, in effect, the computing system 110 may select one of the indication codes from the set of candidate indication codes. The computing system 110 may update claim data with the selected indication code in lieu of the missing one or store the generated object in a suitable database or data repository. The stored data object may be accessible, for example, by a claim processor or a claim-processing system. Additionally or alternatively, the stored data object may be accessible by a system that compiles use data for the medication, e.g., to enable market intelligence.

### Example Data Flow

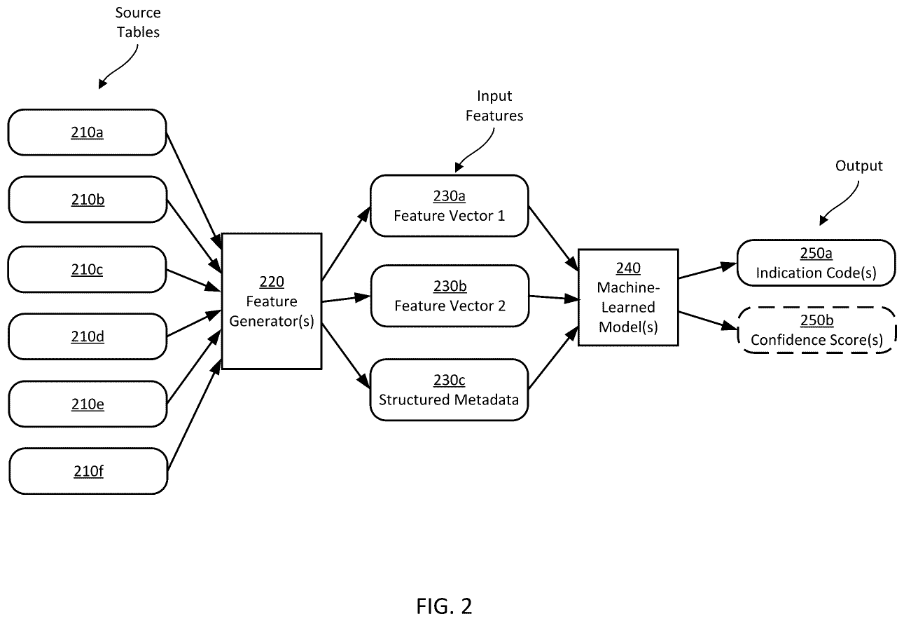

FIG. 2 depicts an example data flow 200 for selecting an indication code 250a associated with a claim from the set of candidate indication codes. The data flow 200 may include generating a confidence score 250b associated with the selected indication code.

The input data may include source tables 210a-f from a variety of data sources. In other embodiments, the data input may include structured strings, or other suitable data objects. The source tables 210a-f may come from databases (e.g., patient data 142, provider data 144, medication data 146) stored at the medical data server 140 and from claim data 132 (e.g., tabulated by the computing system 110 or pre-tabulated at the claim data source 130). Generally, input source tables may include any suitable number of tables (e.g., 2, 3, 4, 5, 6, 8, 10, 12, 15, 20, etc.). In one example, tables 210a-f include a claim transaction table 210a, a claim rebate table 210b, a member table 210c, a prescriber table 210d, a medication table 210e, and an auxiliary table 210f.

The claim transaction table 210a may include prescription information including the medicine name or another identifier, a dosage, a duration of the prescription, an identifier of a prescriber, an identifier of a member (e.g., the individual for whom the medicine is prescribed and the claim filed), and any other suitable data. The claim transaction table 210a may be configured to include an indication, but the indication may be missing or incorrect, as described above. The claim rebate table 210b may include information about any rebates claimed, which may be included in a metadata feature because rebates can be indication-specific. The tables 210a, b may be based on claim data 132 from the claim data source 130.

The member table 210c may be based on a query of patient data 142 using the identifier of the member from the claim transaction table 210a and may include information about the individual for whom the medicine is prescribed. The information about the individual in the member table 210c may include previous prescriptions for the same or equivalent medication and respective indications. The prescriber table 210d may be based on a query of provider data 144 using the identifier of the prescriber from the claim transaction table 210a and may include information about the medical service provider who prescribed the medication. The information about the prescriber in the prescriber table 210d may include previous prescriptions written by the medical service provider for the same or equivalent medication. The medication table 210e may be based on a query of the medication data 146 using the identifier of the medication from the claim transaction table 210a and may include a current list of approved indications for the medication. In some embodiments, the medication table 210e includes frequencies with which the medication is provided for different indications.

One or more feature generators 220 (e.g., feature generator 115) may generate input features 230a-c for the machine learned model(s) 240 based on the data contained in the source tables 210a-e. The one or more feature generators 220 included in and/or implemented by the feature generator 115. Input features 230a-c include a first feature vector 230a, a second feature vector 230b, and a structured metadata feature 230c. The first feature vector 230a, as discussed above, may be based on frequencies with which the provider prescribed the medication for a variety of indications. The feature generator(s) 220 may construct first feature vector 230a based on the data in the prescriber table 210d. The second feature vector 230b, as discussed above, may be based on frequencies with which different indications were used in the prescription of the medication for the individual for whom the medication is prescribed. The feature generator(s) 220 may construct first feature vector 230a based on the data in the member table 210c.

The feature generator(s) 220 may construct the structured metadata feature 230c based on one or more of the tables 210a-e. The structured metadata feature 230c may be an array, a formatted string, or any other suitable data object. In some embodiments, the structured metadata feature 230c includes indications of age and/or gender of the individual for whom the medication is prescribed (e.g., the member associated with the member table). The age in the structured metadata feature 230c may be identified by a bin into which the age of the member falls. For example, the bins may include: under 18, 18 to 24, 25 to 34, 35 to 44, 45 to 54, 55 to 64, 65 to 74, 75 to 84, 85 or older. The age bins need not be uniform and may have any suitable size. The granularity of the age bins may be tuned based on training of the machine-learned models to optimize performance. In other embodiments, a birth date, a birth year, and/or an age rounded to the nearest year may be used as an indication of age in the structured metadata feature 230c. In some embodiments, the structured metadata feature 230c includes indications of medication dosage and/or quantity of medication dispensed, based on the claim transaction table 210a, for example.

In some embodiments, the feature generator(s) 220 may include a transformer-based machine-learned model configured to generate a text-based feature, as a part of the structured metadata feature 230c or as an additional feature. For example, the transformer-based model may be a summarizer configured to summarize provider notes and/or detect certain terms or keywords.

The machine-learned model(s) 240 are configured for processing the input features 230a-c. In some embodiments, at least some of the machine-learned models are implemented by the processor(s) 112 of the computing system 110 running the indication predictor 116. Additionally or alternatively, the indication predictor 116 is configured to pre-process at least some of the input features 230a-c and/or send the input features to one or more machine-learned models executed, for example, on a remote server. That is, at least some of the machine-learned models 240 need not run on the same computing system as the feature generator(s) 220.

The machine-learned models 240 may include an ensemble model comprising a stack of base models. The stack of base models may include tree-based model and/or a neural network. The tree-based model may be based on eXtreme Gradient Boosting (XG Boost), Light Gradient Boosting Machine (LGBM), a random forest, and/or any other suitable framework or architecture. The neural network may be a convolutional neural network (CNN), a recurrent neural network (RNN), or any other suitable architecture. In some embodiments, the machine-learned model(s) include a transformer-based model configured to process a text-based feature generated by the feature generator(s) 220. In some embodiments, each of the machine-learned model(s) 240 is dedicated to a respective one of the input features 230a-c. More generally, each one of the machine-learned model(s) may be configured to take as input any combination of the input features 230a-c. The ensemble model may combine the outputs of the base models in any suitable manner to generate the indication code(s) 250a and, in some embodiments, associated confidence score(s) 250b. In one embodiment, for example, one machine-learned model may operate on the first feature vector 230a and the second feature vector 230b to generate a first output, while another machine-learned model operates on the structured metadata feature 230c and additionally operates on the output of the model operating on the feature vectors 230a, b. As another example, the outputs of a first model operating on the feature vectors 230a, b and a second model operating on the structured metadata feature 230c may serve as inputs to a third machine-learned model or combined in another suitable manner.

Training data for supervised learning techniques for the machine-learned models 240 may include labeled data with features generated from previous claim data and known indications. In some examples, training data may include data samples with individuals who have no prescription history for a particular medication and/or providers who have no prescription history for a particular medication. Such “hard” samples may be relegated to validation data. In some embodiments, the computing system 110 performs the training. In other embodiments, however, another computing system performs at least part of the training, for at least some of the machine-learned models.

### Example Feature Generator and Ensemble Model

FIG. 3 depicts an example feature generator module 310 which may be implemented by the computing system 110. The example feature generator module 310 may include the feature generator(s) 220 and/or may be implemented at least in part by the feature generator 115 algorithm. The feature generator module 310 includes several submodules: a vector generator 312, a metadata assembler 314, and a text generator 316.

The vector generator 312 is configured to generate two feature vectors (e.g., feature vectors 230a, b). The two feature vectors each belong to an N-dimensional vector space, where N is the number of candidate indications for a given medication. The number of candidate indications may be the list of all indications approved by a regulatory agency (e.g., the Food and Drug Administration) for the medication. In other examples, the candidate indications may be based on indications from prior claims.

Each of the N component magnitudes of the first of the two feature vectors may be indicative of a respective frequency with which the medication has been prescribed by a medical service provider or a prescriber for a respective indication. The component magnitudes may be normalized by vector generator 312 to add to unity. Alternatively, the component magnitudes may be normalized by vector generator 312 such that the total vector magnitude is unity, or vector generator 312 may use any other suitable normalization technique.

For the first feature vector, each of the magnitudes may be proportional to the number of times that the provider or prescriber prescribed the medication for the respective indication in a span of time. The span of time may encompass the career of the medical service provider. In other embodiments, the span of time may be set to one, two, five, 10 years or any other suitable duration. In some embodiments, the vector component magnitudes are weighted to reflect recency of prescription. A more recent prescriptions may be weighted more than an older one. For example, the vector generator 312 may process 20 prescriptions by a given provider for a given medication to generate vector component magnitudes for four possible indications (N=4). Over the considered time span of five years, the provider may have prescribed 10 times for the first indication with two prescriptions per year. For the second indication, the provider may have prescribed five times, all of them three years ago. For the third indication, the provider may have prescribed five times, with three in the last year and two in the prior year. For the fourth indication, the provider may have written no prescriptions. In this example, the recency weights may be 1, 0.8, 0.6, 0.4, and 0.2 for the last five years, respectively, with the most recent one having the weight of 1. Pre-normalization, the magnitudes m1, m2, m3, and m4 of the first feature vector may be computed as follows:

$$m 1 = 2 + 2 \times 0.8 + 2 \times 0.6 + 2 \times 0.4 + 2 \times 0.2 = 6$$

$$m 2 = 5 * 0.6 = 3$$

$$m 3 = 3 + 2 * 0.8 = 4.6$$

$$m 4 = 0$$

For the second feature vector, each of the magnitudes may be proportional to the number of times that an individual has been prescribed the medication for the respective indication in a span of time. The span of time may encompass the duration of the medical history of the individual. In other embodiments, the span of time may be set to one, two, five, 10 years or any other suitable duration. In some embodiments, the vector component magnitudes are weighted to reflect recency of prescription. A more recent prescriptions may be weighted more than an older one. For example, the vector generator 312 may process five prescriptions for the individual for a given medication to generate vector component magnitudes for four possible indications (N=4). Over the considered time span of five years, the individual may have been prescribed three times for the first indication, two, three and four years ago, respectively, and twice for the second indication over the course of the last year. As above, the recency weights may be 1, 0.8, 0.6, 0.4, and 0.2 for the last five years, respectively, with the most recent one having the weight of 1. Pre-normalization, the magnitudes m1, m2, m3, and m4 of the second feature vector may be computed as follows:

$$m 1 = 0.6 + 0.4 + 0.2 = 1.2$$

$$m 2 = 2$$

$$m 3 = 0$$

$$m 4 = 0$$

In other examples, the recency weights need not be linear for either the first or the second feature vector, and may reflect any suitable function, such as exponential decay.

The metadata assembler 314 may be configured to assemble data from input tables (e.g., input tables 210a-e) or another suitable data source into a structured metadata feature (e.g., structured metadata feature 230c). The structured metadata may include age, gender, medical conditions, allergies, and/or any other suitable structured data describing the individual to whom the medication is prescribed. The output of the metadata assembler 314 may be a formatted string, a file, or any other suitable data object. For example, the output of the metadata assembler 314 may be a JavaScript Object Notation (JSON) file. In another example, each field of the structured metadata may be represented by a number encoding a value and providing a suitable input for a machine-learned model.

The text generator 316 may be configured to generate a text feature based on text and/or other unstructured data associated with a claim. The text generator 316 may use a transformer-based model to summarize and/or describe the unstructured data.

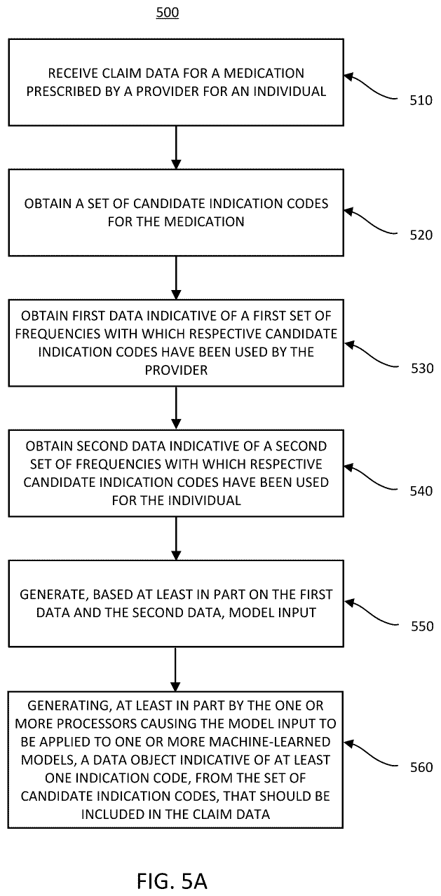

FIG. 4 depicts an example ensemble machine-learned model 410 having a tree-based base model 412, a neural network base model 414, and, in some embodiments, a text analysis base model 416. The text analysis base model 416 may be based on a transformer architecture. The ensemble machine-learned model 410 includes a stack combiner 418 to combine the outputs of the base models 412-416. For example, component base models 412 and 414 may each output a subset of candidate indications with respective confidence scores. The stack combiner 418 may select an indication from the candidate indications based on the highest average or weighted average confidence score. More generally, the ensemble machine-learned model 410 may use any suitable algorithm reflecting qualities of outputs of the component base models to generate an indication.

### Example Method for Selecting an Indication

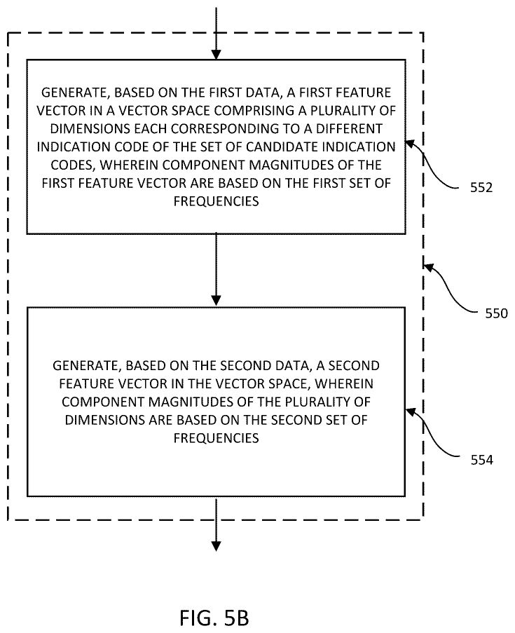

FIGS. 5A and 5B depict flow diagrams representing an example computer-implemented method 500 that implements techniques of this disclosure. FIG. 5A depicts blocks 510-550 and 560. FIG. 5B depicts block 550 of the method 500 in more detail, according to one embodiment. The method 500 is implemented by one or more processors, e.g., by processor(s) 112 of the computing system 110 when executing instructions of feature generator 115 and possibly indication predictor 116.

At block 510, the method 500 includes receiving (e.g., by the computing system 110 and from the claim data source 130) claim data for a medication prescribed by a medical service provider (e.g., prescriber) for an individual (e.g., member of an insurance plan). At block 520, the method 500 includes obtaining (e.g., by the computing system 110) a set of candidate indication codes (or, simply, indications) for the medication. The computing set of candidate indications may be obtained from a data source pertaining to the medication, such as the medication data 146 and/or the medication table 210e.

At block 530, the method 500 includes obtaining (e.g., by the computing system 110) first data indicative of a first set of frequencies with which respective candidate indication codes have been used by the provider. The provider data 144 and/or the prescriber table 210d can serve as the source of the first set of frequencies. The data may include, for example, all the prescriptions that the provider wrote for the medication, the times when the prescriptions were written, and the indications.

At block 540, the method 500 includes obtaining (e.g., by the computing system 110) second data indicative of a second set of frequencies with which respective candidate indication codes have been used for the individual for whom the prescription is written. The patient data 142 and/or the member table 210c can serve as the source of the second set of frequencies. The data may include, for example, all the prescriptions that the individual (insurance member associated with the claim) received for the medication, the times when the prescriptions were written, and the indications.

At block 550, the method 500 includes generating (e.g., using the feature generator 115, the feature generator(s) 220, and/or the feature generator module 310) based at least in part on the first data and the second data, model input.

As depicted in FIG. 5B, the block 530 includes sub-blocks 552 and 554. At sub-blocks 552 and 554, the method 500 includes generating a first feature vector and a second feature vector, each belonging to an N-dimensional vector space comprising a plurality of dimensions each corresponding to a different indication code of the set of candidate indication codes. At sub-block 552, the method 500 includes generating, based on the first data, the first feature vector, wherein magnitudes of the first feature vector that correspond to the plurality of dimensions are based on the first set of frequencies. At sub-block 554, the method includes generating, based on the first data, the second feature vector, wherein magnitudes of the second feature vector that correspond to the plurality of dimensions are based on the second set of frequencies. The vector generator 312 may generate the first and the second feature vectors, as described with reference to FIGS. 1-3.

The method 500 may also include generating additional features. For example, the method 500 may include generating the structured metadata feature by extracting structured metadata from the claim data and/or other sources. The structured metadata feature may include age and/or gender of the individual to whom the medication is prescribed, the dose and quantity of prescribed medication, etc. The method 500 may further include generating a text feature, as discussed with reference to FIG. 3.

Returning now to FIG. 5A, at block 560, the method 500 includes generating (e.g., creating, modifying, updating, etc.) a data object indicative of at least one indication code, from the set of candidate indication codes, that should be included in the claim data. To generate the data object at block 560, the method 500 includes causing the model input to be applied to one or more machine-learned models, as described, for example, with reference to FIGS. 1, 2 and 4. In some embodiments, the one or more machine-learned models include a plurality of base models, as described, for example, with reference to FIG. 4.

Of course, it is to be appreciated that the actions of the method 500 may be performed any suitable number of times, and that the actions described in reference to the method 500 may be performed in any suitable order.

### EXAMPLES

Example 1. A computer-implemented method comprising: receiving, by one or more processors, claim data for a medication prescribed by a provider for an individual; obtaining, by the one or more processors, a set of candidate indication codes for the medication; obtaining, by the one or more processors, first data indicative of a first set of frequencies with which respective candidate indication codes have been used by the provider; obtaining, by the one or more processors, second data indicative of a second set of frequencies with which respective candidate indication codes have been used for the individual; generating, by the one or more processors and based at least in part on the first data and the second data, model input, wherein generating the model input includes: generating, based on the first data, a first feature vector in a vector space comprising a plurality of dimensions each corresponding to a different indication code of the set of candidate indication codes, wherein component magnitudes of the first feature vector that correspond to the plurality of dimensions are based on the first set of frequencies; and generating, based on the second data, a second feature vector in the vector space, wherein component magnitudes of the second feature vector that correspond to the plurality of dimensions are based on the second set of frequencies; and generating, at least in part by the one or more processors causing the model input to be applied to one or more machine-learned models, a data object indicative of at least one indication code, from the set of candidate indication codes, that should be included in the claim data.

Example 2. The computer-implemented method of example 1, wherein generating the model input further includes generating an additional feature by extracting structured metadata from the claim data.

Example 3. The computer-implemented method of example 2, wherein the structured metadata includes one or more characteristics of the individual, the one or more characteristics including one or both of gender and age.

Example 4. The computer-implemented method of example 2 or 3, wherein the structured metadata includes one or more characteristics indicative of dosage or quantity of the medication.

Example 5. The computer-implemented method of any one of examples 1-4, wherein the one or more machine-learned models include an ensemble model that includes a plurality of base models.

Example 6. The computer-implemented method of example 5, wherein the plurality of base models includes one or both of a tree-based model and a neural network.

Example 7. The computer-implemented method of any one of examples 1-6, wherein the data object indicative of the indication code includes a respective confidence score.

Example 8. The computer-implemented method of any one of examples 1-7, wherein: generating the first feature vector includes determining respective vector component magnitudes that are in proportion to different frequencies of the first set of frequencies, and normalizing the first vector; and generating the second feature vector includes determining respective vector component magnitudes that are in proportion to different frequencies of the second set of frequencies, and normalizing the second vector.

Example 9. A system comprising: one or more processors; and one or more memories storing processor-executable instructions that, when executed by the one or more processors, cause the one or more processors to perform operations comprising the computer-implemented method of any one of examples 1-8.

Example 10. One or more non-transitory, computer-readable media storing processor-executable instructions that, when executed by one or more processors, cause the one or more processors to perform operations comprising the computer-implemented method of any one of examples 1-8.

### ADDITIONAL CONSIDERATIONS

Throughout this specification, components, operations, or structures described as a single instance may be implemented as multiple instances. Although individual operations of one or more methods (or processes, techniques, routines, etc.) are illustrated and described as separate operations, two or more of the individual operations may be performed concurrently or otherwise in parallel, and nothing requires that the operations be performed in the order illustrated. Structures and functionality (e.g., operations, steps, blocks) presented as separate components in example configurations may be implemented as a combined structure, functionality, or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, operations, blocks, or instructions. These may constitute and/or be implemented by software (e.g., code embodied on a non-transitory, machine-readable medium), hardware, or a combination thereof. In hardware, the routines, etc., may represent tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware component that operates to perform certain operations as described herein.

In various embodiments, a hardware component may be implemented mechanically or electronically. For example, a hardware component may comprise dedicated circuitry or logic that is permanently configured (e.g., as a special-purpose processor, such as a field programmable gate array (FPGA) or an application-specific integrated circuit (ASIC)) to perform certain operations. A hardware component may also or instead comprise programmable logic or circuitry (e.g., as encompassed within one or more general-purpose processors and/or other programmable processor(s)) that is temporarily configured by software to perform certain operations.

Accordingly, the term “hardware component” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where the hardware components include a general-purpose processor configured using software, the general-purpose processor may be configured as respective different hardware components at different times. Software may accordingly configure a processor, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple of such hardware components exist contemporaneously, communications may be achieved through signal transmission (e.g., over appropriate circuits and buses) that connect the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information).

As noted above, the various operations of example methods (or processes, techniques, routines, etc.) described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions. The components referred to herein may, in some example embodiments, comprise processor-implemented components.

Moreover, each operation of processes illustrated as logical flow graphs may represent a sequence of operations that can be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

The terms “coupled” and “connected,” along with their derivatives, may be used. In particular embodiments, “connected” may be used to indicate that two or more elements are in direct physical or electrical contact with each other, although the context in the description may dictate otherwise when it is apparent that two or more elements are not in direct physical or electrical contact. “Coupled” may mean that two or more elements are in direct physical or electrical contact. However, “coupled” may also mean that two or more elements are not in direct contact with each other, yet still co-operate, transmit between, or interact with each other.

An algorithm may be considered to be a self-consistent sequence of acts or operations leading to a desired result. These include physical manipulations of physical quantities. Usually, though not necessarily, these quantities take the form of electrical, magnetic, or optical signals capable of being stored, transferred, combined, compared, and otherwise manipulated. These signals are commonly referred to as bits, values, elements, symbols, characters, terms, numbers, flags, or the like. It should be understood, however, that all of these and similar terms are to be associated with the appropriate physical quantities and are merely convenient labels applied to these quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “some embodiments,” “one embodiment,” “an embodiment,” “in some examples,” or variations thereof means that a particular element, feature, structure, characteristic, operation, or the like described in connection with the embodiment is included in at least one embodiment, but not every embodiment necessarily includes the particular element, feature, structure, characteristic, operation, or the like. Different instances of such a reference in various places in the specification do not necessarily all refer to the same embodiment, although they may in some cases. Moreover, different instances of such a reference may describe elements, features, structures, characteristics, operations, or the like be combined in any manner as an embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless the context of use clearly indicates otherwise, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term “set” is intended to mean a collection of elements and can be a null set (i.e., a set containing zero elements) or may comprise one, two, or more elements. A “subset” is intended to mean a collection of elements that are all elements of a set, but that does not include other elements of the set. A first subset of a set may comprise zero, one, or more elements that are also elements of a second subset of the set. The first subset may be said to be a subset of the second subset if all the elements of the first subset are elements of the second subset, while also being a subset of the set. However, if all the elements of the second subset are also elements of the first subset (in addition to all the elements of the first subset being elements of the second subset), the first subset and the second subset are a single subset/not distinct.

For the purposes of the present disclosure, the term “a” or “an” entity refers to one or more of that entity. As such, the terms “a” or “an”, “one or more”, and “at least one” can be used interchangeably herein unless explicitly contradicted by the specification using the word “only one” or similar. For example, “a first element” may functionally be interpreted as “a first one or more elements” or a “first at least one element.” Unless otherwise apparent from the context of use, reference in the present disclosure to a same set of “one or more processors” (or a same “plurality of processors,” etc.) performing multiple operations can encompass implementations in which performance of the operations is divided among the processor(s) in any suitable way. For example, “generating, by one or more processors, X; and generating, by the one or more processors, Y” can encompass: (1) implementations in which a first subset of the processors (e.g., in a first computing device) generates X and an entirely distinct, second subset of the processors (e.g., in a different, second computing device) independently generates Y; (2) implementations in which one or more or all of the processor(s) (e.g., one or multiple processors in the same device, or multiple processors distributed among multiple devices) contribute to the generation of X and/or Y; and (3) other variations. This may similarly be applied to any other component or feature similarly recited (e.g., as “a component”, “a feature”, “one or more components”, “one or more features”, “a plurality of components”, “a plurality of features”). Moreover, the performance of certain of the operations may be distributed among the one or more components, not only residing within a single machine, but deployed across a number of machines. The set of components may be located in a single geographic location (e.g., within a home environment, an office environment, a cloud environment). In other example embodiments, the set of components may be distributed across two or more geographic locations. Further, “a machine-learned model”, equivalent terms (e.g., “machine learning model,” “machine-learning model,” “machine-learned component”, “artificial intelligence”, “artificial intelligence component”), or species thereof (e.g., “a large language model”, “a neural network”) may include a single machine-learned model or multiple machine-learned models, such as a pipeline comprising two or more machine-learned models arranged in series and/or parallel, an agentic framework of machine-learned models, or the like.

An “artificial intelligence” or “artificial intelligence component” may comprise a machine-learned model. A machine-learned model may comprise a hardware and/or software architecture having structural hyperparameters defining the model's architecture and/or one or more parameters (e.g., coefficient(s), weight(s), biase(s), activation function(s) and/or action function type(s) in examples where the activation function and/or function type is determined as part of training, clustering centroid(s)/medoid(s), partition(s), number of trees, tree depth, split parameters) determined as a result of training the machine-learned model based at least in part on training hyperparameters (e.g., for supervised, semi-supervised, and reinforcement learning models) and/or by iteratively operating the machine-learned model according to the training hyperparameters(e.g., for unsupervised machine-learned models).

In some examples, structural hyperparameter(s) may define component(s) of the model's architecture and/or their configuration/order, such as, for example, the configuration/order specifying which input(s) are provided to one component and which output(s) of that component are provided as input to other component(s) of the machine-learned model; a number, type, and/or configuration of component(s) per layer; a number of layers of the model; a number and/or type of input nodes in an input layer of the model; a number and/or type of nodes in a layer; a number and/or type of output nodes of an output layer of the model; component dimension (e.g., input size versus output size); a number of trees; a maximum tree depth; node split parameters; minimum number of samples in a leaf node of a tree; and/or the like. The component(s) of the model may comprise one or more activation functions and/or activation function type(s) (e.g., gated linear unit (GLU), such as a rectified linear unit (ReLU), leaky RELU, Gaussian error linear unit (GELU), Swish, hyperbolic tangent), one or more attention mechanism and/or attention mechanism types (e.g., self-attention, cross-attention), nodes and split indications and/or probabilities in a decision tree, and/or various other component(s) (e.g., adding and/or normalization layer, pooling layer, filter). Various combinations of any these components (as defined by the structural hyperparameter(s)) may result in different types of model architectures, such as a transformer-based machine-learned model (e.g., enreviewer-only model(s), enreviewer-dereviewer model(s), dereviewer-only models, generative pre-trained transformer(s) (GPT(s))), neural network(s), multi-layer perceptron(s), Kolmogorov-Arnold network(s), clustering algorithm(s), support vector machine(s), gradient boosting machine(s), and/or the like. The structural parameters and components a machine-learned model comprises may vary depending on the type of machine-learned model.

Training hyperparameter(s) may be used as part of training or otherwise determining the machine-learned model. In some examples, the training hyperparameter(s), in addition to the training data and/or input data, may affect determining the parameter(s) of the target machine-learned model. Using a different set of training hyperparameters to train two machine-learned models that have the same architecture (i.e., the same structural hyperparameters) and using the same training data may result in the parameters of the first machine-learned model differing from the parameters of the second machine-learned model. Despite having the same architecture and having been trained using the same training data, such machine-learned models may generate different outputs from each other, given the same input data. Accordingly, accuracy, precision, recall, and/or bias may vary between such machine-learned models.

In some examples, training hyperparameter(s) may include a train-test split ratio, activation function and/or activation function type (e.g., in examples like Kolmogorov-Arnold networks (KANs) where the activation function type is determined as part of training from an available set of activation functions and/or limits on the activation function parameters specified by the training hyperparameters), training stage(s) (e.g., using a first set of hyperparameters for a first epoch of training, a second set of hyperparameters for a second epoch of training), a batch size and/or number of batches of data in a training epoch, a number of epochs of training, the loss function used (e.g., L1, L2, Huber, Cauchy, cross entropy), the component(s) of the machine-learned model that are altered using the loss for a particular batch or during a particular epoch of training (e.g., some components may be “frozen,” meaning their parameters are not altered based on the loss), learning rate, learning rate optimization algorithm type (e.g., gradient descent, adaptive, stochastic) used to determine an alteration to one or more parameters of one or more components of the machine-learned model to reduce the loss determined by the loss function, learning rate scheduling, and/or the like.

In some examples, the structural hyperparameters and/or the training hyperparameters may be determined by a hyperparameter optimization algorithm or based on user input, such as a software component written by a user or generated by a machine-learned model. The machine-learned model may include any type of model configured, trained, and/or the like to generate a prediction output for a model input. In some examples, any of the logic, component(s), routines, and/or the like discussed herein may be implemented as a machine-learned model.

The machine-learned model may include one or more of any type of machine-learned model including one or more supervised, unsupervised, semi-supervised, and/or reinforcement learning models. Training a machine-learned model may comprise altering one or more parameters of the machine-learned model (e.g., using a loss optimization algorithm) to reduce a loss. Depending on whether the machine-learned model is supervised, semi-supervised, unsupervised, etc. this loss may be determined based at least in part on a difference between an output generated by the model and ground truth data (e.g., a label, an indication of an outcome that resulted from a system using the output), a cost function, a fit of the parameter(s) to a set of data, a fit of an output to a set of data, and/or the like. In some examples, determining an output by a machine-learned model may comprise executing a set of inference operations executed by the machine-learned model according to the target machine-learned model's parameter(s) and structural hyperparameter(s) and using/operating on a set of input data.

Moreover, any discussion of receiving data associated with an individual that may be protected, confidential, or otherwise sensitive information, is understood to have been preceded by transmitting a notice of use of the data to a computing device, account, or other identifier (collectively, “identifier”) associated with the individual, receiving an indication of authorization to use the data from the identifier, and/or providing a mechanism by which a user may cause use of the data to cease or a copy of the data to be provided to the user.

Upon reading this disclosure, those of skill in the art will appreciate still additional alternative structural and functional designs through the principles disclosed herein. Therefore, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The patent claims at the end of this patent application are not intended to be construed under 35 U.S.C. § 112(f) unless traditional means-plus-function language is expressly recited, such as “means for” or “step for” language being explicitly recited in the claim(s).

What is claimed is:

1. A computer-implemented method comprising:
receiving, by one or more processors, claim data for a medication prescribed by a provider for an individual;
obtaining, by the one or more processors, a set of candidate indication codes for the medication;
obtaining, by the one or more processors, first data indicative of a first set of frequencies with which respective candidate indication codes have been used by the provider;
obtaining, by the one or more processors, second data indicative of a second set of frequencies with which respective candidate indication codes have been used for the individual;
generating, by the one or more processors and based at least in part on the first data and the second data, model input, wherein generating the model input includes:
generating, based on the first data, a first feature vector in a vector space comprising a plurality of dimensions each corresponding to a different indication code of the set of candidate indication codes, wherein component magnitudes of the first feature vector that correspond to the plurality of dimensions are based on the first set of frequencies; and
generating, based on the second data, a second feature vector in the vector space, wherein component magnitudes of the second feature vector that correspond to the plurality of dimensions are based on the second set of frequencies; and
generating, at least in part by the one or more processors causing the model input to be applied to one or more machine-learned models, a data object indicative of at least one indication code, from the set of candidate indication codes, that should be included in the claim data.

2. The computer-implemented method of claim 1, wherein generating the model input further includes generating an additional feature by extracting structured metadata from the claim data.

3. The computer-implemented method of claim 2, wherein the structured metadata includes one or more characteristics of the individual, the one or more characteristics including one or both of gender and age.

4. The computer-implemented method of claim 2, wherein the structured metadata includes one or more characteristics indicative of dosage or quantity of the medication.

5. The computer-implemented method of claim 1, wherein the one or more machine-learned models include an ensemble model that includes a plurality of base models.

6. The computer-implemented method of claim 5, wherein the plurality of base models includes one or both of a tree-based model and a neural network.

7. The computer-implemented method of claim 1, wherein the data object indicative of the at least one indication code includes a respective confidence score.

8. The computer-implemented method of claim 1, wherein:
generating the first feature vector includes determining respective vector component magnitudes that are in proportion to different frequencies of the first set of frequencies, and normalizing the first feature vector; and
generating the second feature vector includes determining respective vector component magnitudes that are in proportion to different frequencies of the second set of frequencies, and normalizing the second feature vector.

9. A system comprising:
one or more processors;
one or more memories storing processor-executable instructions that, when executed by the one or more processors, cause the one or more processors to perform operations comprising:
receiving claim data for a medication prescribed by a provider for an individual;
obtaining a set of candidate indication codes for the medication;
obtaining first data indicative of a first set of frequencies with which respective candidate indication codes have been used by the provider;
obtaining second data indicative of a second set of frequencies with which respective candidate indication codes have been used for the individual;
generating, based at least in part on the first data and the second data, model input, wherein generating the model input includes:
generating, based on the first data, a first feature vector in a vector space comprising a plurality of dimensions each corresponding to a different indication code of the set of candidate indication codes, wherein component magnitudes of the first feature vector that correspond to the plurality of dimensions are based on the first set of frequencies; and
generating, based on the second data, a second feature vector in the vector space, wherein component magnitudes of the second feature vector that correspond to the plurality of dimensions are based on the second set of frequencies; and
generating, at least in part by the one or more processors causing the model input to be applied to one or more machine-learned models, a data object indicative of at least one indication code, from the set of candidate indication codes, that should be included in the claim data.

10. The system of claim 9, wherein generating the model input is based at least in part on generating an additional feature by extracting structured metadata from the claim data.

11. The system of claim 10, wherein the structured metadata includes one or more characteristics of the individual, the one or more characteristics including one or both of gender and age.

12. The system of claim 10, wherein the structured metadata includes one or more characteristics indicative of dosage or quantity of the medication.

13. The system of claim 9, wherein the one or more machine-learned models include an ensemble model that includes a plurality of base models.

14. The system of claim 13, wherein the plurality of base models includes one or both of a tree-based model and a neural network.

15. The system of claim 9, wherein the data object indicative of the at least one indication code includes a respective confidence score.

16. The system of claim 9, wherein:
generating the first feature vector includes determining respective vector component magnitudes that are in proportion to different frequencies of the first set of frequencies, and normalizing the first feature vector; and
generating the second feature vector includes determining respective vector component magnitudes that are in proportion to different frequencies of the second set of frequencies, and normalizing the second feature vector.

17. One or more non-transitory, computer-readable media storing processor-executable instructions that, when executed by one or more processors, cause the one or more processors to perform operations comprising:
receiving claim data for a medication prescribed by a provider for an individual;
obtaining a set of candidate indication codes for the medication;
obtaining first data indicative of a first set of frequencies with which respective candidate indication codes have been used by the provider;
obtaining second data indicative of a second set of frequencies with which respective candidate indication codes have been used for the individual;
generating, based at least in part on the first data and the second data, model input, wherein generating the model input includes:
generating, based on the first data, a first feature vector in a vector space comprising a plurality of dimensions each corresponding to a different indication code of the set of candidate indication codes, wherein magnitudes of the first feature vector that correspond to the plurality of dimensions are based on the first set of frequencies; and
generating, based on the second data, a second feature vector in the vector space, wherein magnitudes of the second feature vector that correspond to the plurality of dimensions are based on the second set of frequencies; and
generating, at least in part by the one or more processors causing the model input to be applied to one or more machine-learned models, a data object indicative of at least one indication code, from the set of candidate indication codes, that should be included in the claim data.

18. The one or more non-transitory, computer-readable media of claim 17, wherein generating the model input is based at least in part on generating a third feature by extracting structured metadata from the claim data.

19. The one or more non-transitory, computer-readable media of claim 18, wherein the structured metadata includes one or more characteristics of the individual, the one or more characteristics including one or both of gender and age.

20. The one or more non-transitory, computer-readable media of claim 18, wherein the structured metadata includes one or more characteristics indicative of dosage or quantity of the medication.
